(19) United States
(12) Patent Application Publication
(10) Pub. No.: US 2021/0393696 A1
(43) Pub. Date: Dec. 23, 2021

(54) COMPOSITIONS FOR IMPROVING VACCINE SAFETY AND EFFICACY AND METHODS OF USE THEREOF

(71) Applicant: Kansas State University Research Foundation, Manhattan, KS (US)

(72) Inventor: Megan Niederwerder, Manhattan, KS (US)

(21) Appl. No.: 17/309,201

(22) PCT Filed: Nov. 6, 2019
(86) PCT No.: PCT/US2019/060100

§ 371 Date: May 6, 2021

Related U.S. Application Data
(60) Provisional application No. 62/756,326, filed on Nov. 6, 2018.

(51) Int. Cl. A61K 35/24; A61K 39/12; A61K 39/145; A61K 39/187; A61K 39/245; A61K 39/02; A61K 39/102; A61K 39/112; A61K 39/05; A61P 31/12; A61K 35/741; A61K 39/39

(52) U.S. Cl. CPC A61K 35/24; A61K 39/12; A61K 39/145; A61K 39/187; A61K 39/245; A61K 39/099; A61K 2039/55594; A61K 39/0275; A61K 39/05; A61P 31/12; A61K 35/741; A61K 39/39; A61K 39/102

(57) ABSTRACT

The present disclosure provides methods and compositions for reducing the incidence, severity, and/or duration of at least one sign of respiratory infection. The methods include the steps of administering a composition comprising gastrointestinal microbiota and an immunogenic composition to an animal in need thereof.

### CROSS REFERENCE TO RELATED APPLICATIONS

This application claims the benefit of U.S. Provisional Application Ser. No. 62/756,326, which is hereby incorporated by reference in its entirety.

### STATEMENT REGARDING FEDERALLY SPONSORED RESEARCH & DEVELOPMENT

None

### BACKGROUND OF THE DISCLOSURE

The field of the disclosure relates generally to improving the safety and efficacy of vaccinations. More particularly, the field of the disclosure relates to improving an animal's microbiome. Still more particularly, the field of the disclosure relates to improving an animal's microbiome through fecal microbiome transplantation. Even more particularly, the field of the disclosure relates to improving the overall health of an animal, the safety of vaccinations, and the efficacy of vaccinations by administering fecal microbiome transplantation around the same time as vaccination.

### BRIEF DESCRIPTION OF THE DISCLOSURE

There are two main categories of respiratory diseases in pigs. The first category includes diseases that affect large numbers of pigs and may be serious but they are of limited duration. The second category includes diseases that persist in a large number of pigs for indefinite periods. Diseases in the first category can be costly, but the losses are limited rather than ongoing. They include swine influenza (see Swine Influenza), classical swine fever (see Classical Swine Fever), the pneumonic forms of pseudorabies (see Pseudorabies), porcine circovirus-associated disease (see Porcine Circovirus Diseases), and porcine reproductive and respiratory syndrome (see Porcine Reproductive and Respiratory Syndrome). The most significant pathogens in the second category are Salmonella, Haemophilus parasuis, Bordetella bronchiseptica, Pasteurella and especially Pasteurella multocida, and Actinobacillus pleuropneumoniae. The negative economic impact of these diseases result from adverse and uneven effect on growth rate, decreased feed efficiency, and additional costs of drugs including medicated feed.

Many commercial vaccines are available to combat respiratory pathogens, but their efficacy can vary due to a multitude of factors including timing, dosage, and health status of the pig prior to vaccination.

In the present disclosure, the terms immunogenic composition and vaccine are used interchangeably.

The present disclosure provides compositions and methods for improving vaccine performance such that clinical signs or signs of infection by one or more of the respiratory pathogens are reduced in severity, incidence, duration in individual pigs or groups of pigs. In some forms of the disclosure, at least one clinical sign of infection is reduced in severity, incidence, or duration in pigs receiving at least one administration of the composition in comparison to groups of pigs or individual pigs that do not receive an administration of a composition of the disclosure. In individual pigs, the comparison may be in the average number of clinical signs related to respiratory infection, or it may be in the average severity of one or more clinical signs of infection of a group of pigs, or it may be in the duration of one or more clinical signs of infection of a pig or group of pigs. For groups of pigs, the comparison may be between the averages of the groups for severity, duration, or incidence of one or more clinical signs. In some forms, the severity of clinical signs is reduced at least 10, 15, 20, 25, 30, 35, 40, 45, 50, 55, 60, 65, 70, 75, 80, 85, 90, 95, 96, 97, 98, 99, or even 100% when comparing an individual pig or group of pigs that did receive at least one administration of a composition of the disclosure to a group of pigs that did not receive an administration of a composition of the disclosure. In some forms, the duration of clinical signs is reduced at least 10, 15, 20, 25, 30, 35, 40, 45, 50, 55, 60, 65, 70, 75, 80, 85, 90, 95, 96, 97, 98, 99, or even 100% when comparing an individual pig or group of pigs that did receive at least one administration of a composition of the disclosure to a group of pigs that did not receive an administration of a composition of the disclosure. In some forms, the incidence of clinical signs is reduced at least 10, 15, 20, 25, 30, 35, 40, 45, 50, 55, 60, 65, 70, 75, 80, 85, 90, 95, 96, 97, 98, 99, or even 100% when comparing an individual pig or group of pigs that did receive at least one administration of a composition of the disclosure to a group of pigs that did not receive an administration of a composition of the disclosure.

Clinical signs or signs of respiratory infection include fever, pneumonia, lethargy, failure to thrive, gross and histological lung lesions, transient pyrexia, dyspnea and tachypnea, labored breathing, pyrexia, cough, asthma, lameness, shivering, and disorder in the respiratory tract, and anorexia.

In some forms, vaccinated pigs that also receive at least one administration of a composition according to the disclosure will have increased weight gain in comparison to vaccinated pigs that do not also receive at least one administration of a composition of the disclosure.

In some forms, the administration of a composition of the present disclosure will result in increased diversity of the microbiome of the animal. In some forms, there will be 1, 2, 3, 4, 5, 6, 7, 8, 9, 10, 11, 12, 13, 14, 15, or more numbers of species and/or families of bacteria than in animals that did not receive at least one administration of a composition of the disclosure.

In some forms, the administration of a composition of the present disclosure will result in an increase in the prevalence of one or more types of Streptococcaceae and/or Ruminococcaceae bacteria in the microbiome of an animal.

In some forms, the administration of a composition of the present disclosure will result in a decrease in the prevalence of Methanobacteriaceae in the microbiome of an animal.

In some forms, the administration of a composition of the present disclosure will result in needing less vaccine to achieve the same protection level. For example, if a 2 ml dose of vaccine results in 99% protection against clinical signs of infection, and 1 ml dose of vaccine together with at least one administration of a composition of the present disclosure will also result in 99% protection, the vaccine dosage can be reduced 50% when the composition is also being administered. Preferably, the amount of vaccine necessary to achieve a desired level of protection can be reduced at least 5, 10, 15, 20, 25, 30, 35, 40, 45, 50, 55, 60, 65, 70, 75, 80, 85, 90% or more.

In some forms, the administration of a composition of the present disclosure results in a slower increase in viremia in pigs after vaccination and challenge with a respiratory virus. In some forms, the slower increase in viremia is in comparison to pigs that did not receive an administration of the composition but received the same vaccination. In some forms, the composition comprises one or more microorganisms that are found in the gastrointestinal microbiome of an animal to each pig. In some forms, the vaccine or immunogenic composition is effective for reducing the severity, incidence, or duration of at least one clinical sign of at least one respiratory infection in a pig to each pig. In some forms, the immunogenic composition is effective against a pathogen selected from the group consisting of Porcine Reproductive and Respiratory Syndrome Virus (PRRSV), porcine circovirus (PCV), swine influenza virus, classical swine fever, pseudorabies virus, Salmonella, Haemophilus parasuis, Bordetella bronchiseptica, Pasteurella, Actinobacillus pleuropneumoniae, and any combination thereof. Preferably, the viremia is reduced at least 10, 15, 20, 25, 30, 35, 40, 45, 50, 55, 60, 65, 70, 75, 80, 85, 90, 95, 96, 97, 98, 99, or even 100% in comparison to a pig or group of pigs that received the administration of immunogenic composition but did not receive the administration of the composition. In some forms, the composition is administered more than one time. In some forms, the composition is administered at least 1, 2, 3, 4, 5, 6, 7, 8, 9, 10, 11, 12, 13, 14, 15, or more time before and at least 1, 2, 3, 4, 5, 6, 7, 8, 9, 10, 11, 12, 13, 14, 15, or more times after the administration of the immunogenic composition. In some forms, the microorganisms in the composition are from an animal that was healthy. In some forms, the composition comprises at least 2 log CFU/ml/dose of microorganism when the microorganism is a bacteria, or at least 2 TCID50/ml/dose when the microorganism is a virus.

In some forms, the present disclosure provides a method of increasing the efficacy of an immunogenic composition comprising the steps of: administering a composition comprising one or more microorganisms that are found in the gastrointestinal microbiome of an animal to each pig; and administering at least one immunogenic composition effective for reducing the severity, incidence, or duration of at least one clinical sign of at least one respiratory infection in a pig to each pig, wherein the efficacy of the immunogenic composition is increased in comparison to a pig or group of pigs that received the administration of the immunogenic composition but did not receive the administration of the composition. In some forms, the immunogenic composition is effective against a pathogen selected from the group consisting of Porcine Reproductive and Respiratory Syndrome Virus (PRRSV), porcine circovirus (PCV), swine influenza virus, classical swine fever, pseudorabies virus, Salmonella, Haemophilus parasuis, Bordetella bronchiseptica, Pasteurella, Actinobacillus pleuropneumoniae, and any combination thereof. In some forms, the efficacy of the immunogenic composition is increased by at least 10, 20, 30, 40, 50, 60, 70, 80, 90, or even 100% in comparison to a pig or group of pigs that received the administration of immunogenic composition but did not receive the administration of the composition. In some forms, the composition is administered more than 1, 2, 3, 4, 5, 6, 7, 8, 9, 10, 11, 12, 13, 14, 15, or more times. In some forms, the composition is administered at least 1, 2, 3, 4, 5, 6, 7, 8, 9, 10, 11, 12, 13, 14, 15, or more times before and at least 1, 2, 3, 4, 5, 6, 7, 8, 9, 10, 11, 12, 13, 14, 15, or more times after the administration of the immunogenic composition. In some forms, the microorganisms in the composition are from an animal that was healthy. In some forms, the microorganisms in the composition are from the same species as the animal receiving the administration. In some forms, the composition comprises at least 2 log CFU/ml/dose of microorganism when the microorganism is a bacteria, or at least 2 TCID50/ml/dose when the microorganism is a virus.

In some forms, the administration of a composition of the present disclosure results in a more prolonged clearance of viremia in pigs after vaccination and challenge with a respiratory virus. In some forms, the slower increase in viremia is in comparison to pigs that did not receive an administration of the composition but received the same vaccination.

In all forms, the comparison is between pigs that have been vaccinated against the same pathogens.

In some forms, the composition comprises fecal material and/or fecal microbiota from one or more healthy animals, preferably from the same species to whom the composition will be administered.

In another aspect of the present disclosure, a composition for reducing the incidence of or severity of clinical signs associated with or caused by a pathogen is provided. In some forms, the composition is administered to a subject susceptible to infection by the pathogen prior to contact with the pathogen. In some forms, the composition is administered to a subject susceptible to infection by the pathogen after contact with or infection by the pathogen. In some forms, the pathogen is infective and causes clinical signs in a mammal. In some forms, the mammal is a pig. In some forms, the pathogen is selected from the group consisting of PRRSV, PCV2, and any combination thereof. In some forms, the composition is administered via a bolus, chewable product, oral drench, nasal drench, placement on feed (feed additive or top dress), placement in water, or placed on the mammary gland of a sow for subsequent consumption by suckling animals. In some forms, the composition is administered at any time. In some forms, the composition is administered 1-2 days after birth. In other forms, the composition is administered up to or at the time of weaning. In other forms, the composition is administered immediately after weaning in the early nursery period. In some forms, the composition is administered prior to vaccination. In some forms, the composition is administered both before and after vaccination. In still other forms, the composition is administered after the presence of a pathogen is discovered in a herd or group of animals. In still other forms, the composition is administered on a repeated basis to members of the group used for breeding purposes. In some forms, the composition comprises one or more microorganisms including bacteria or viruses that are found in the gastrointestinal microbiome. In some forms, the composition comprises one or more microorganisms that are found in the microbiome of an animal that is resistant to infection by a pathogen capable of causing clinical signs in animals of the same species as the animal that is resistant. In some forms, the composition comprises one or microorganisms that is found in greater numbers or in a higher proportion relative to an animal that is not resistant to infection by the pathogen. In some forms, the microorganism is selected from the group consisting of a member of a family selected from the group consisting of Intrasporangiaceae, Veillonellaceae, Lachnospiraceae, Ruminococcaceae, Streptococcaceae, and any combination thereof (List 1). In some forms, the microorganism is selected from the group consisting of Intrasporangiaceae bacterium JGI 0001002-M5, Rhinovirus B, Escherichia coli, Streptococcus equi and any combination thereof (List 2). In some forms, the microorganism is selected from the group consisting of one or more of the microorganisms provided in the List 3 below:

List 3:

| OTUID | taxonomy | taxonomy | Highly_Abundant_In |
|---|---|---|---|
| 10 | o_Erysipelotrichales | f_Erysipelotrichaceae | D0_Unaffected |
| 47 | o_Clostridiales | f_Ruminococcaceae | D0_Unaffected |
| 55 | o_Clostridiales | f_Veillonellaceae | D0_Unaffected |
| 14 | o_Clostridiales | f_Ruminococcaceae | D0_Unaffected |
| 155 | o_Bacteroidales | f_Prevotellaceae | D0_Unaffected |
| 252 | o_Clostridiales | f_Lachnospiraceae | D0_Unaffected |
| 42 | o_Clostridiales | f_Ruminococcaceae | D0_Unaffected |
| 394 | o_Clostridiales | f_Ruminococcaceae | D0_Unaffected |
| 254 | o_Clostridiales | f_Ruminococcaceae | D0_Unaffected |
| 270 | o_Bacteroidales | f_No Assigned Family | D0_Unaffected |
| 262 | o_RF39 | f_No Assigned Family | D0_Unaffected |
| 426 | o_Clostridiales | f_Lachnospiraceae | D0_Unaffected |
| 162 | o_Bacteroidales | f_No Assigned Family | D0_Unaffected |
| 645 | o_Clostridiales | f_Clostridiaceae | D0_Unaffected |
| 260 | o_Clostridiales | f_No Assigned Family | D0_Unaffected |
| 431 | o_Bacteroidales | f_Prevotellaceae | D0_Unaffected |
| 497 | o_Erysipelotrichales | f_ Erysipelotrichaceae | D0_Unaffected |
| 347 | o_Bacteroidales | f_No Assigned Family | D0_Unaffected |
| 334 | o_Coriobacteriales | f_Coriobacteriaceae | D0_Unaffected |
| 1674 | o_Clostridiales | f_Ruminococcaceae | D0_Unaffected |
| 638 | o_Erysipelotrichales | f_Erysipelotrichaceae | D0_Unaffected |
| 306 | o_Clostridiales | f_Ruminococcaceae | D0_Unaffected |
| 655 | o_Erysipelotrichales | f_Erysipelotrichaceae | D0_Unaffected |
| 450 | o_Clostridiales | f_Ruminococcaceae | D0_Unaffected |
| 1607 | o_Clostridiales | f_Lachnospiraceae | D0_Unaffected |
| 244 | o_Clostridiales | f_Lachnospiraceae | D0_Unaffected |
| 1734 | o_Clostridiales | f_No Assigned Family | D0_Unaffected |
| 276 | o_Clostridiales | f_Lachnospiraceae | D0_Unaffected |
| 557 | o_Clostridiales | f_Ruminococcaceae | D0_Unaffected |

In some forms, the microorganism is selected from the group consisting of one or more of the microorganisms provided in List 4 below wherein “p” denotes Phylum, “c” denotes Class; “o” denotes Order, “f” denotes Family, “g” denotes Genus, and “s” denotes Species:

List 4:
_Firmicutes; c_Clostridia; o_Clostridiales; f_Veillonellaceae; g_; s_
p_Firmicutes; c_Clostridia; o_Clostridiales; f_Ruminococcaceae; g_Oscillospira; s_
p_Firmicutes; c_Clostridia; o_Clostridiales; f_Lachnospiraceae; g_Dorea; s_
p_Firmicutes; c_Clostridia; o_Clostridiales; f_Veillonellaceae; g_; s_
p_Firmicutes; c_Clostridia; o_Clostridiales; f_Veillonellaceae; g_Megasphaera; s_
p_Bacteroidetes; c_Bacteroidia; o_Bacteroidales; f_S24-7; g_; s_
p_Firmicutes; c_Clostridia; o_Clostridiales; f_Ruminococcaceae; g_; s_
p_Firmicutes; c_Clostridia; o_Clostridiales; f_Lachnospiraceae; g_; s_
p_Firmicutes; c_Clostridia; o_Clostridiales; f_Lachnospiraceae; g_; s_
p_Actinobacteria; c_Coriobacteriia; o_Coriobacteriales; f_Coriobacteriaceae; g_; s_
p_Bacteroidetes; c_Bacteroidia; o_Bacteroidales; f_; g_; s_
p_Firmicutes; c_Clostridia; o_Clostridiales; f_Ruminococcaceae; g_; s_
p_Bacteroidetes; c_Bacteroidia; o_Bacteroidales; f_; g_; s_
p_Firmicutes; c_Clostridia; o_Clostridiales; f_Veillonellaceae; g_Megasphaera; s_
p_Firmicutes; c_Clostridia; o_Clostridiales; f_Ruminococcaceae; g_; s_

In some forms, the composition comprises one or more microorganisms from one or more of lists 1, 2, 3, and 4. In some forms, the composition comprises at least 2, 3, 4, 5, 6, 7, 8, 9, or more microorganisms. In some forms, the composition comprises one or more microorganisms from a phylum selected from the group consisting of Firmicutes, Bacteroidetes, Actinobacteria, and any combination thereof. In some forms, the composition comprises one or more microorganisms from a class selected from the group consisting of Clostridia, Bacteroidia, Coriobacteriia, and any combination thereof. In some forms, the composition comprises one or more microorganisms from an order selected from the group consisting of Erysipelotrichales, Clostridiales, Bacteroidales, RF39, Coriobacteriales, and any combination thereof. In some forms, the composition comprises one or more microorganisms from a family selected from the group consisting of Intrasporangiaceae, Veillonellaceae, Lachnospiraceae, Ruminococcaceae, Erysipelotrichaceae, Prevotellaceae, Clostridiaceae, Streptococcaceae, Coriobacteriaceae, S24-7, and any combination thereof. In some forms, the composition comprises one or more organisms from a genus selected from the group consisting of Megasphaera, Oscillospira, Dorea, and any combination thereof. In some forms, the composition comprises one or more organisms from a species selected from the group consisting of Intrasporangiaceae bacterium JGI 0001002-M5, Rhinovirus B, and any combination thereof. In some forms, the composition comprises at least two or more microorganisms independently and respectively selected from the phylums, classes, orders, families, geniuses, and species described above. In some forms, the composition includes, or is administered with a prebiotic. In some forms, the composition is administered with or includes a component selected from the group consisting of a preservative, stabilizer, antibiotic, and any combination thereof.

In some forms, the composition comprises at least 2 log CFU/ml/dose of microorganism when the microorganism is a bacteria, more preferably between 2-9 log CFU/ml/dose, more preferably between 3-8 log CFU/ml/dose, still more preferably between 5-7 log CFU/ml/dose. In some forms, the composition comprises at least 2 TCID50/ml/dose when the microorganism is a virus, more preferably between 2-10 TCID50/ml/dose, even more preferably between 2-8 TCID50/ml/dose, still more preferably between 2-6 TCID50/ml/dose, even more preferably between 2-4 TCID50/ml/dose.

In some forms, the composition is made by obtaining microorganisms from the gastrointestinal microbiota of an animal, preferably a healthy animal, processing the fecal microbiota therein, and concentrating the fecal microbiota. In some forms, the fecal microbiota can be frozen and stored.

The microbiome, or collection of microorganisms in the gastrointestinal tract, are critical for development of immunity and digestion of nutrients. Modulating the microbiome should be considered an alternative to antibiotics for reducing morbidity and mortality due to respiratory disease, enhancing growth and improving vaccine safety and efficacy.

In some forms, the composition and the vaccine are included in a kit, wherein the composition is as described above and the vaccine is a commercially-available vaccine. The kit further comprises instructions for administering the composition and the vaccine to animals in need thereof.

### BRIEF DESCRIPTION OF THE DRAWINGS

FIG. 1 is a schematic representation of the experimental design to investigate the effects of FMT on PRRSV challenge in PRRS-vaccinated and nonvaccinated pigs;

FIG. 2A is a graph illustrating PRRS viremia in nonvaccinated pigs transplanted with fecal microbiota or saline with 10% glycerol. Data is shown as the mean±standard deviation log₁₀ copies/PCR reaction for each group. Statistical significance or trends (*p<0.05; ‡p≤0.1) are shown for each day based on unpaired t-test analysis;

FIG. 2B is a graph illustrating PRRS viremia in vaccinated pigs transplanted with fecal microbiota or saline with 10% glycerol. Data is shown as the mean±standard deviation log₁₀ copies/PCR reaction for each group. Statistical significance or trends (*p<0.05; ‡p≤0.1) are shown for each day based on unpaired t-test analysis;

FIG. 3A is a graph illustrating microscopic lung lesion scores in nonvaccinated pigs with or without fecal microbiota transplantation at 42 days post-challenge with PRRSV. Lung lesions are scored by a blinded board certified pathologist reviewing histopathology slides of sections from each lung lobe. Scores include 0: no lesions, 1: mild and multifocal interstitial pneumonia with <50% lobe involvement, 2: mild to moderate and multifocal interstitial pneumonia with 50-75% lobe involvement, 3: moderate to severe and multifocal interstitial pneumonia with 50-75% lobe involvement, and 4: severe diffuse interstitial pneumonia with >75% lobe involvement. No significant difference was detected between nonvaccinated pigs with and without FMT (p=0.7403; Mann Whitney U test);

FIG. 3B is a graph illustrating microscopic lung lesion scores in vaccinated pigs with and without fecal microbiota transplantation at 42 days post-challenge with PRRSV. Lung lesions are scored by a blinded board certified pathologist reviewing histopathology slides of sections from each lung lobe. Scores include 0: no lesions, 1: mild and multifocal interstitial pneumonia with <50% lobe involvement, 2: mild to moderate and multifocal interstitial pneumonia with 50-75% lobe involvement, 3: moderate to severe and multifocal interstitial pneumonia with 50-75% lobe involvement, and 4: severe diffuse interstitial pneumonia with >75% lobe involvement. No significant difference was detected between vaccinated pigs with and without FMT (p=0.2245; Mann Whitney U test).

FIG. 4A is a photograph showing no microscopic lung lesions in pigs 42 days after challenge with virulent PRRSV;

FIG. 4B is a photograph showing mild and multifocal interstitial pneumonia with <50% lobe involvement in pigs 42 days after challenge with virulent PRRSV;

FIG. 4C is a photograph showing mild to moderate and multifocal interstitial pneumonia with 50-75% lobe involvement in pigs 42 days after challenge with virulent PRRSV;

FIG. 4D is a photograph showing moderate to severe and multifocal interstitial pneumonia with 50-75% lobe in pigs 42 days after challenge with virulent PRRSV;

FIG. 5A is a graph illustrating weight gain in FMT and control pigs without PRRS MLV vaccination prior to virulent PRRSV challenge. Data is shown as mean absolute weights±standard deviation as measured weekly throughout the 28 day post-vaccination and 42 day post-challenge periods. A trend towards significance (‡p=0.086, unpaired t-test) was detected on 14 days post-challenge in the nonvaccinated group with transplanted pigs having higher absolute mean weights; and

FIG. 5B is a graph illustrating weight gain in FMT and control pigs with PRRS MLV vaccination prior to virulent PRRSV challenge. Data is shown as mean absolute weights±standard deviation as measured weekly throughout the 28 day post-vaccination and 42 day post-challenge periods. No significant difference in weight was detected in vaccinated pigs which received the FMT when compared to those which received saline.

### DETAILED DESCRIPTION OF THE DISCLOSURE

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

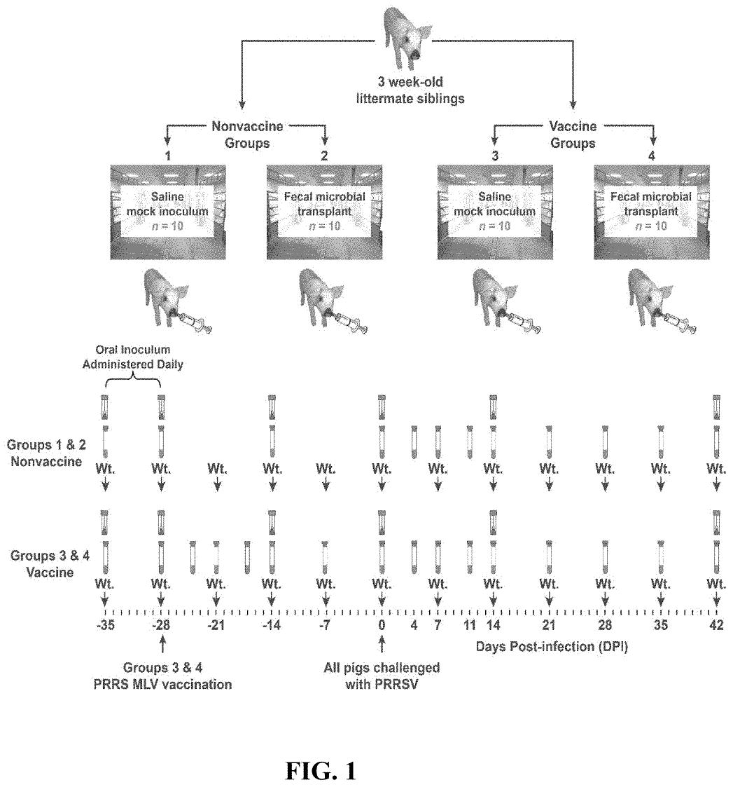

Vaccine Study: Fecal microbiota transplantation was performed in pigs prior to vaccination with a PRRS modified live virus (MLV) vaccine and subsequent challenge with virulent PRRSV. First, feces was collected from 2 high health donors as previously described in PCT/US2018/033910. The exact same material was utilized to assess the effects of FMT on PRRS-only challenge in vaccinated and nonvaccinated pigs in the current study. The experimental design is summarized in FIG. 1. Forty high-health commercial pigs (average age 18.6±0.5 days) were obtained from a single source herd negative for PRRSV. Four brother barrows were divided into the four experimental groups (n=10/group) and balanced by weight upon arrival. Mean weight across all four groups was 11.45 lbs. Each group was housed in an individual pen and all pens were housed in 1 environmentally controlled room under biosafety-level 2 conditions. Biosecurity protocols were implemented between pens to prevent cross-contamination of gut microbes and PRRS viruses. Pigs were given access to food and water ad libitum. Upon arrival, FMT Vaccinated and FMT Nonvaccinated pigs received the fecal transplant material daily for 7 days (average 3.5 ml/day) while the Control Vaccinated and Control Nonvaccinated pigs received sterile saline with 10% glycerol for 7 days (average 3.5 ml/day). The volume of FMT material administered in the current study was less than the volume administered in PCT/US2018/033910, which was 5 ml/pig/day. The FMT had been prepared from this previous study (PCT above) using feces collected from two older sows with specific high health characteristics, including high parity (≥9), large litters with high numbers of born alive piglets, low pre-weaning mortality, no history of fetal mummification, no fecal parasites, and no antibiotic treatment within the year prior to collection. The fecal microbiota were processed, concentrated, and stored at −80° C. using a protocol adapted from the human FMT literature. Prior to administration, the FMT is thawed for 2 hrs on ice. After 7 days of fecal transplantation or mock saline transplantation, all pigs in the Vaccinated groups received a 2 mL dose of a commercial PRRS MLV vaccine (Ingelvac PRRS MLV; Boehringer Ingelheim Animal Health) administered intramuscularly according to the vaccine label instructions. At 28 days post-vaccination, all pigs in all four groups were challenged with virulent PRRSV administered as a 2 mL dose containing 105 TCID50 PRRSV in MEM. The 2-mL dose was split, with 1 mL administered intranasally and the remaining 1 mL administered intramuscularly. Pigs were followed for 42 days post-challenge with PRRSV (FIG. 1). Pigs were evaluated by a veterinarian or veterinary assistant daily. Standardized health evaluation protocols were used to score clinical disease post-infection. Blood samples were collected from all pigs on −35, −28, 0, 4, 7, 11, 14, 21, 28, 35, and 42 dpi. Additionally, blood samples were collected from the vaccinated groups on −24, −21, −17, −14, and −7 dpi. PRRSV viremia was quantified using the EZ-PRRSV MPX 4.0 Real Time RT-PCR Target-Specific Reagents (Tetracore). Individual body weights were collected on −35, −28, −21, −14, −7, 0, 7, 14, 21, 28, 35, and 42 dpi. Average daily gain (ADG) was calculated as the change in weight over the change in time. All pigs were humanely euthanized at 42 dpi and complete necropsies were performed by a board certified pathologist. Microscopic lung lesion severity was estimated using 0 to 4 scoring systems as previously described.

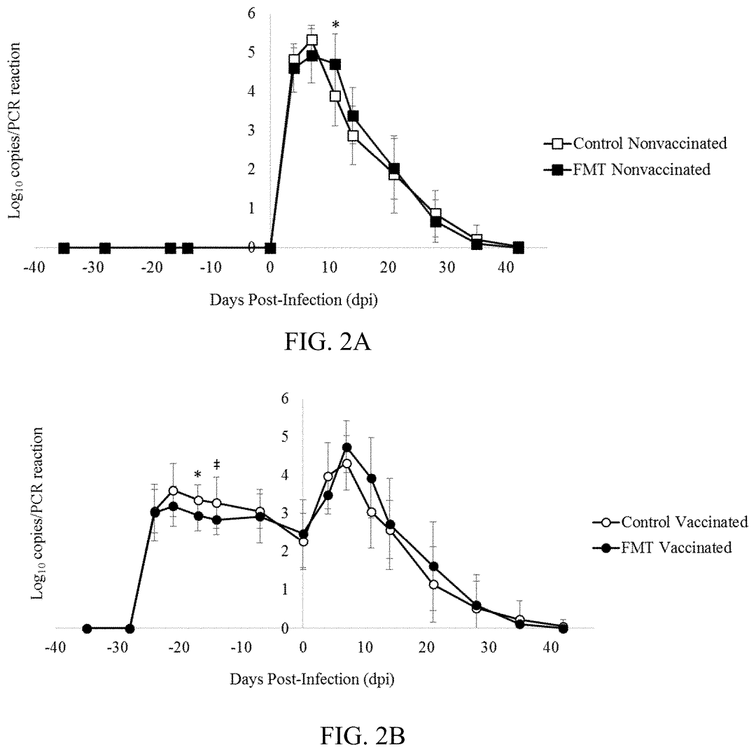

Overall, clinical signs were mild throughout the study and no mortalities occurred post-challenge with virulent PRRSV. PRRS virus replication was compared between control and vaccinated groups with and without FMT (FIG. 2). In the vaccinated groups, replication of the PRRS MLV vaccine was measured between 0 to 28 days post-vaccination. Fecal transplantation seemed to reduce replication of the PRRS MLV vaccine. Specifically, on −21, −17 and −14 days post-infection, the transplanted group had numerically less PRRS virus detectable in the serum. The total vaccine virus replication during this period (7 to 14 days post-vaccination) was calculated as the area under the curve (Table 1).

TABLE 1
Total vaccine virus replication between 7 to 14 days post-vaccination in pigs
immunized with a PRRS MLV vaccine with and without fecal microbiota
transplantation*

| Control Vaccinated | | FMT Vaccinated | |
|---|---|---|---|
| Pig | AUC | Pig | AUC |
| 30 | 18.69 | 1 | 15.63 |
| 18 | 20.02 | 39 | 18.21 |
| 9 | 21.95 | 13 | 20.34 |
| 22 | 22.71 | 21 | 20.90 |
| 37 | 23.98 | 17 | 21.43 |
| 2 | 24.79 | 27 | 21.68 |
| 26 | 25.28 | 32 | 21.74 |
| 15 | 26.14 | 31 | 21.79 |
| 6 | 27.57 | 8 | 23.71 |
| 35 | 27.67 | 7 | 24.21 |
| Mean‡ | 23.88 | Mean‡ | 20.96 |
| SEM | 0.9593 | SEM | 0.7925 |

*Total virus replication calculated as the area under the curve (AUC, log₁₀ copies/PCR reaction) for individual pigs between 7, 11, and 14 days post-vaccination
‡Significant difference between means, p = 0.0308 (student's unpaired t test)

For control pigs, mean AUC was 23.88±0.9593 log 10 copies/PCR reaction with a range between 18.69 and 27.67. For transplanted pigs, mean AUC was 20.96±0.7925 log 10 copies/PCR reaction with a range between 15.63 and 24.21. The difference between the two groups with regards to total vaccine virus replication over this 1 week period was statistically significant (p=0.0308, unpaired t-test). Additionally, there was a trend towards significance with the FMT pigs having lower total vaccine virus replication as measured by the area under the curve between 4 and 21 days post-vaccination (p=0.0798, student's unpaired t-test). When comparing individual days, on −17 dpi, transplanted pigs had significantly lower PRRS viremia (p=0.04) and on −14 dpi, transplanted pigs had a trend towards reduced vaccine virus replication (p=0.1). Overall, a reduction in the replication of the PRRS MLV vaccine increases the safety of the vaccine and reduces vaccine virus shedding. This would be beneficial due to the risk of shedding vaccine virus to nonvaccinated pigs and the potential effects of vaccine virus replication on production parameters (i.e., morbidity and growth).

Post-challenge with virulent PRRSV, the vaccinated FMT pigs had a slower increase in PRRSV viremia followed by a more prolonged clearance. On 11 dpi, FMT vaccinated pigs had a trend towards increased virulent PRRS virus detection in the blood (p=0.08). In nonvaccinated pigs, no PRRSV was detected in transplanted or control pigs prior to virulent PRRSV challenge, confirming that no exposure to the PRRS MLV vaccine virus occurred and biosecurity was maintained. Post-challenge with virulent PRRSV, the nonvaccinated FMT pigs had a lower peak followed by more prolonged viral clearance as shown by a significant increase in PRRSV detection in the blood at 11 dpi (p=0.04).

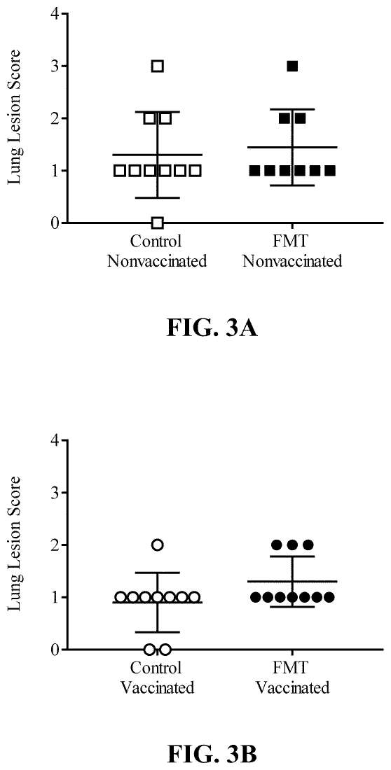

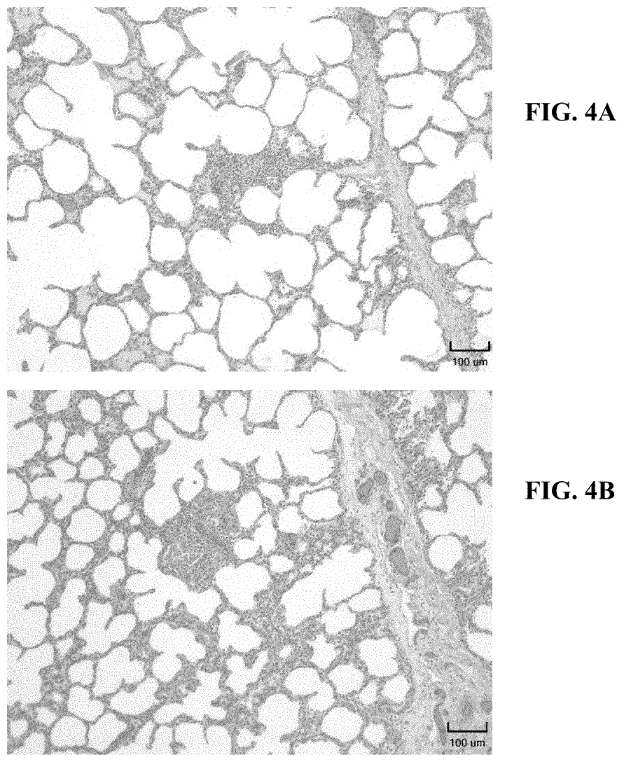

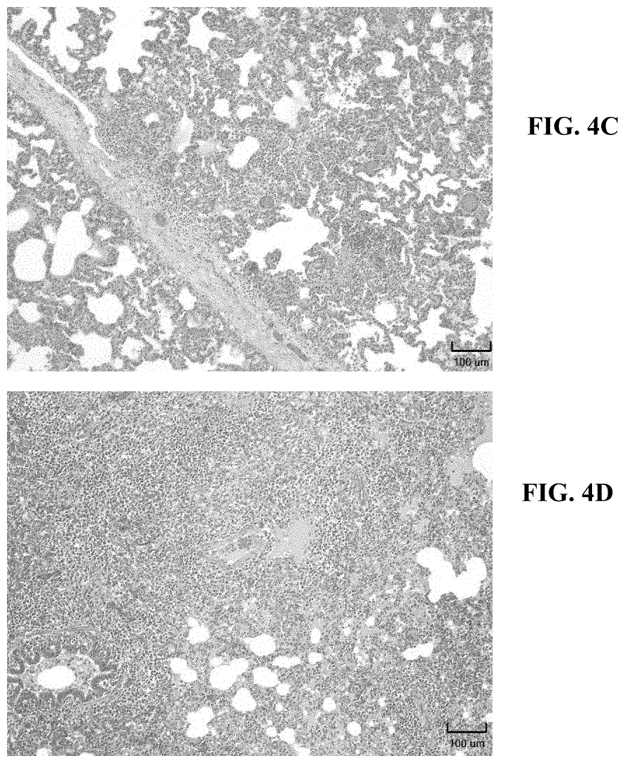

At the conclusion of the 42-day post infection period, gross necropsies were completed on all pigs by a blinded board-certified veterinary pathologist. Lung sections were collected from each lung lobe and fixed in formalin. H&E stained histopathology slides of lung tissue were reviewed and scored for severity of interstitial pneumonia (FIG. 3). Scores include 0: no lesions, 1: mild and multifocal interstitial pneumonia with <50% lobe involvement, 2: mild to moderate and multifocal interstitial pneumonia with 50-75% lobe involvement, 3: moderate to severe and multifocal interstitial pneumonia with 50-75% lobe involvement, and 4: severe diffuse interstitial pneumonia with >75% lobe involvement. Examples of lung lesions representing the 4 scores that were detected in the current study are shown in FIG. 4. The majority of pigs had some degree of interstitial pneumonia; however, no significant difference was detected between the transplanted and control pigs which received PRRS MLV vaccination (p=0.2245; Mann Whitney U test) or between the transplanted and control pigs without vaccination (p=0.7403; Mann Whitney U test).

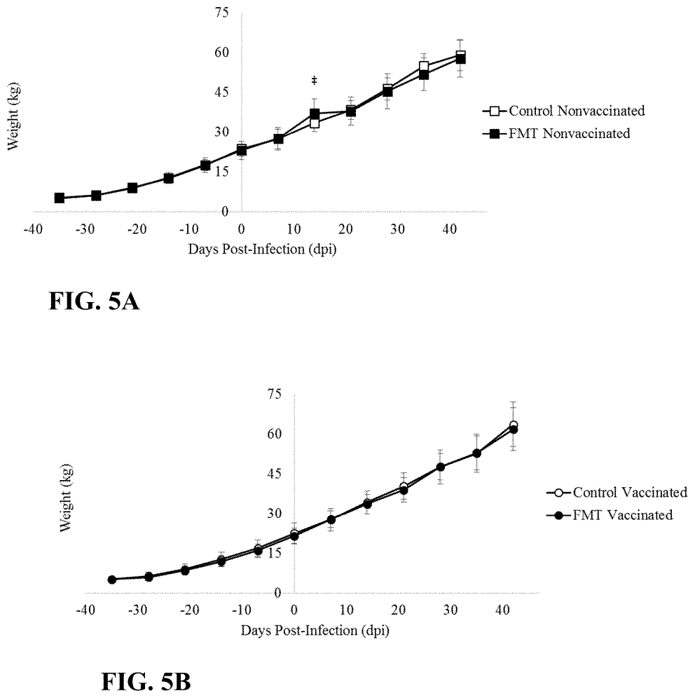

Overall, weight gain was similar between the FMT and control pigs in both the vaccinated and nonvaccinated groups (FIG. 5). On 14 dpi, there was a trend towards a significant difference between the absolute weights in the nonvaccinated FMT and control groups. Control pigs had a mean weight of 33.5±3.3 kg compared to a mean weight of 37.2±5.3 kg in the FMT group (p=0.086, student's unpaired t test). No other significant differences or trends towards significance were detected in absolute weights between the four groups.

1. A method for reducing the severity, incidence, or duration of at least one clinical sign of respiratory infection in a pig or group of pigs or increasing the efficacy of an immunogenic composition comprising the steps of:
administering a composition comprising one or more microorganisms that are found in the gastrointestinal microbiome of an animal to each pig; and
administering at least one immunogenic composition effective for reducing the severity, incidence, or duration of at least one clinical sign of at least one respiratory infection in a pig to each pig,
wherein the severity, incidence, or duration of at least one clinical sign of respiratory infection is reduced in comparison to a pig or group of pigs that received the administration of the immunogenic composition but did not receive the administration of the composition, or wherein the efficacy of the immunogenic composition is increased in comparison to a pig or group of pigs that received the administration of the immunogenic composition but did not receive the administration of the composition.

2. The method of claim 1, wherein the immunogenic composition is effective against a pathogen selected from the group consisting of Porcine Reproductive and Respiratory Syndrome Virus (PRRSV), porcine circovirus (PCV), swine influenza virus, classical swine fever, pseudorabies virus, Salmonella, Haemophilus parasuis, Bordetella bronchiseptica, Pasteurella, Actinobacillus pleuropneumoniae, and any combination thereof.

3. The method of claim 1, wherein the severity, incidence, or duration of at least one clinical sign of respiratory infection is reduced at least 10% in comparison to a pig or group of pigs that received the administration of immunogenic composition but did not receive the administration of the composition.

4. The method of claim 1, wherein the composition is administered more than one time.

5. The method of claim 1, wherein the composition is administered at least one time before and at least one time after the administration of the immunogenic composition.

6. (canceled)

7. The method of claim 1, wherein the microorganisms in the composition are from an animal that was healthy.

8. (canceled)

9. The method of claim 1, wherein the diversity of the microbiome of animals receiving the composition is higher than in animals not receiving the composition.

10. The method of claim 1, wherein comprises at least 2 log CFU/ml/dose of microorganism when the microorganism is a bacteria, or at least 2 TCID50/ml/dose when the microorganism is a virus.

11. A method of slowing the increase in viremia in a pig or group of pigs after challenge or infection by a virulent pathogen comprising the steps of:
administering a composition comprising one or more microorganisms that are found in the gastrointestinal microbiome of an animal to each pig; and
administering at least one immunogenic composition effective for reducing the severity, incidence, or duration of at least one clinical sign of at least one respiratory infection in a pig to each pig,
wherein the viremia of the virulent pathogen is reduced in comparison to a pig or group of pigs that received the administration of the immunogenic composition but did not receive the administration of the composition.

12. The method of claim 11, wherein the immunogenic composition is effective against a pathogen selected from the group consisting of Porcine Reproductive and Respiratory Syndrome Virus (PRRSV), porcine circovirus (PCV), swine influenza virus, classical swine fever, pseudorabies virus, Salmonella, Haemophilus parasuis, Bordetella bronchiseptica, Pasteurella, Actinobacillus pleuropneumoniae, and any combination thereof.

13. The method of claim 11, wherein the viremia is reduced at least 10% in comparison to a pig or group of pigs that received the administration of immunogenic composition but did not receive the administration of the composition.

14. The method of claim 11, wherein the composition is administered more than one time.

15. The method of claim 11, wherein the composition is administered at least one time before and at least one time after the administration of the immunogenic composition.

16. (canceled)

17. The method of claim 11, wherein the microorganisms in the composition are from an animal that was healthy.

18. (canceled)

19. The method of claim 11, wherein comprises at least 2 log CFU/ml/dose of microorganism when the microorganism is a bacteria, or at least 2 TCID50/ml/dose when the microorganism is a virus.

20. (canceled)

21. (canceled)

22. (canceled)

23. (canceled)

24. (canceled)

25. (canceled)

26. (canceled)

27. (canceled)

28. (canceled)

29. A kit comprising a composition comprising:
one or more microorganisms that are found in the gastrointestinal microbiome of an animal;
at least one immunogenic composition effective for reducing the severity, incidence, or duration of at least one clinical sign of at least one respiratory infection in a pig to each pig; and
instructions for the administration of both the immunogenic composition and the one or more microorganisms.

30. The kit of claim 29, wherein the immunogenic composition is effective against a pathogen selected from the group consisting of Porcine Reproductive and Respiratory Syndrome Virus (PRRSV), porcine circovirus (PCV), swine influenza virus, classical swine fever, pseudorabies virus, Salmonella, Haemophilus parasuis, Bordetella bronchiseptica, Pasteurella, Actinobacillus pleuropneumoniae, and any combination thereof.

31. The kit of claim 29, wherein the instructions instruct a user to administer the composition more than one time.

32. The kit of claim 29, wherein the instructions in composition is administered at least one time before and at least one time after the administration of the immunogenic composition.

33. (canceled)

34. (canceled)

35. (canceled)

36. The kit of claim 29, wherein the immunogenic composition comprises at least 2 log CFU/ml/dose of microorganism when the microorganism is a bacteria, or at least 2 TCID50/ml/dose when the microorganism is a virus.
